(19) United States
(12) Patent Application Publication
(10) Pub. No.: US 2012/0069950 A1
(43) Pub. Date: Mar. 22, 2012

(54) METHOD AND X-RAY DEVICE FOR CREATING AN X-RAY PROJECTION IMAGE

(75) Inventors: Michael Grasruck, Nuernberg (DE); Karl Stierstorfer, Erlangen (DE)

(73) Assignee: SIEMENS AG, Munich (DE)

(21) Appl. No.: 13/233,071

(22) Filed: Sep. 15, 2011

(30) Foreign Application Priority Data
Sep. 17, 2010 (DE) 102010040963.4

(51) Int. Cl. A61B 6/02; A61B 6/03

(52) U.S. Cl. 378/4; 378/41

(57) ABSTRACT

A method and an X-ray device are disclosed for creating an X-ray projection image of a three-dimensional object under examination and for displaying the projection image. In at least one embodiment, pixel images are recorded from two different perspectives and a projection image is created by overlaying the two pixel images, wherein perspective-related offset of the mapping image pixels is taken into account, pixel-by-pixel, in relation to an imaging surface in the object under examination.

### PRIORITY STATEMENT

The present application hereby claims priority under 35 U.S.C. §119 on German patent application number DE 10 2010 040 963.4 filed Sep. 17, 2010, the entire contents of which are hereby incorporated herein by reference.

### FIELD

At least one embodiment of the invention generally relates to a method for creating an X-ray projection image of a three-dimensional object under examination and displaying the projection image and/or to an X-ray device for carrying out this method.

### BACKGROUND

Similar methods and X-ray devices are generally known. In such cases, to create a projectional X-ray image, referred to as a radiographic image, starting from a focus that is as punctiform as possible, a mostly three-dimensional object under examination is X-rayed and the attenuation of the rays passing through the object under examination is measured behind the object under examination on a radiation-sensitive layer or plane and, in accordance with the measured attenuation, an image of the attenuation of the individual rays and thereby of the X-rayed object under examination is created.

Such projectional X-ray imaging is able to be described very well by geometrical projection: Starting from a focus point, contours are able to be shown on the detector by absorption. Objects which are close to the focus are shown enlarged in accordance with their distance from the detector, objects close to the detector are only slightly enlarged. Since the actual size of an imaged detail is also mostly not known, it is not possible either to draw any conclusions from this about its position. Because of the approximately punctiform focus such images also mostly have a good level of sharp detail regardless of how far from the focus the respective detail displayed is located in the object under examination. The person viewing such an image can thus not see from the image where in the image—in relation to the recording direction—an imaged detail is located or specify which detail is located in front of or behind which other detail. As a rule this demands a significantly more complex tomographic imaging, in which the spatial structure of an object under examination becomes evident.

### SUMMARY

In at least one embodiment of the invention, a simple method is provided for projectional imaging of an object under examination and a simple X-ray apparatus when compared with a CT system, which allow a three-dimensional structure of an object under examination to be detected at least approximately, i.e. enable the three-dimensional location of details of an object under examination to be identified.

Advantageous developments of the invention are the subject matter of subordinate claims.

The inventors have recognized the following:

The concept of depth of field is known from optics with visible light. Objects at the focal point appear sharp, objects lying closer or further away appear unsharp. This can be explained by the fact that rays which originate from one and the same point outside the object plane, because of optical imaging, no longer meet at one point in the image plane. If a viewer has the opportunity to adjust a distance setting on a lens and to view an optical image when doing so, because of the changing position of the depth of field in space he can at least estimate roughly the three-dimensional location occupied by a specific subobject in the image.

A direct transfer of the impression of a specific depth of field to current X-ray imaging is not possible, since the systems do not possess any X-ray optical imaging systems corresponding to the optical lenses. The impression of depth of field is already produced however if rays from a point in the object plane coincide again at a point in the image plane and if this only applies for points of a specific plane. For points outside this plane this no longer applies.

Thus if two or more images of an object under examination are recorded with different focus positions and are overlaid for a predetermined imaging plane intersecting the object under examination (=plane of sharp imaging) so that pixels of the images mapping a specific point in the imaging plane are merged, in the projection image thus produced precisely the objects which lie in the imaging plane appear sharp in the image. In such cases it can basically be said that the imaging plane does not have to be just a flat surface, but can be any given surface in space.

A very similar behavior can be achieved by the spring focus mode. To increase the sampling for CT imaging, the focus on the anode plate is allowed to spring to different positions. In the rebinning which precedes the reconstruction all rays are interpolated on a raster. If the spring focus mode is now also applied in order to increase the resolution for the projection imaging, the necessity arises, when composing the images from different focus positions, to specify the plane for which an interpolation of different spring focus positions is geometrically correct. In this plane points on a line for example are again reproduced as a line in the combined image. Points on a line which do not lie in this plane are represented in the combined image as unsharp, like a line provided with a saw tooth. Thus the entire image effect is comparable with that of depth of field in optics. Objects are only shown sharp in one plane, in all other planes, unsharp contours are produced.

The impression of the presence of a plane of sharpness increases generally the further the focus is shifted for the different projections. Basically such imaging with focus positions widely dispersed from one another corresponds to a recording with a very open aperture and correspondingly flat focus range, while focus positions close together correspond to a very much closed aperture with a correspondingly wide focus range. On the rotating anode this is limited by the plate size and by the deflection able to be realized by the electronics. A further divergence of the different projection angles is offered in the fan beam direction by the use of projections from an extended angle segment, i.e. projections from different, adjacent rotation positions of the gantry. The impression of depth of field can be even further increased with at least one embodiment of this method. Multi-focus systems, as are used for example in scanners with inverse geometry, further also offer the option of increasing the spring width in the z-direction. But with standard geometry scanners too in topo mode projections can be included from different z-positions for calculating and presenting an image with an increased depth of field.

If at least one embodiment of the method described above is used by a viewer such that he is viewing images with differently adjusted imaging planes or planes of sharpness on a screen for example, he can determine at least roughly the location of imaged details of an object under examination. In a development of at least one embodiment of this method this process can also be automated in that, by image analysis and image processing, especially filtering, the associated projections are analyzed as a function of a number of known positions of the imaging plane and sharply-defined objects are filtered out in each case. If finally the objects filtered out in the different planes are combined, a rough three-dimensional image of the object under examination is produced.

According to the basic knowledge described above the inventors propose, in their general description, at least one embodiment of a method for creating an X-ray projection image of a three-dimensional object under examination and displaying the projection image, whereby pixel images are recorded from two different perspectives and a projection image is created by overlaying the two pixel images, in that pixel-by-pixel the perspective-related offset of the image pixels to be imaged is taken into account in relation to an imaging surface in the object under examination.

In concrete terms, at least one embodiment of this method can carry out the following method steps:

First radioscopy of the object under examination by X-rays from a first focus position of a first focus and determination of a first pixel image in an image plane, whereby each pixel reflects the attenuation of an X-ray between first focus and pixel through the object under examination,

Second radioscopy of the unchanged object under examination by X-rays from a second focus position of a second focus and determination of a second pixel image in the image plane, whereby each pixel reflects the attenuation of an X-ray between second focus and pixel through the object under examination,

Creation of at least one projection image from the two pixel images by:

Defining an imaging surface which is arranged between pixel image and the focus position and has a rastering with a plurality of raster points,

Determining a transmission function which in each case overlays the image pixels of the first and second pixel image to form a new projection image, the imaging rays of which pass through the same raster point in the imaging surface,

Overlaying the two pixel images with the transmission function to form a new projection image and

Outputting the projection image as an X-ray image of the object under examination with an output device, for example a screen or printer.

In at least one embodiment, the inventors further propose, to create the two pixel images, that an X-ray tube with spring focus with at least two focus positions spaced apart from one another be used or alternately that a CT system be used, whereby the projectional pixel images are recorded at two focus positions spaced apart from one another.

The inventors further propose a specific embodiment of the inventive method such that:

A number of projection images are created for a number of spaced-apart imaging planes,

In each projection image unsharp image information is filtered out so that only the sharp image information belonging to the respective imaging plane remains behind, and

From the filtered projection images arranged in accordance with their imaging plane, a 3D arrangement of sectional images and/or a 3D presentation is created.

In addition to the inventive method, an X-ray device for creating an X-ray projection image of a three-dimensional object under examination and displaying the projection image is proposed, comprising:

At least one X-ray tube for radioscopy of the object under examination by X-rays from at least two focus positions and creation of at least two pixel images,

A programmable control and processing unit with a memory for storing program code which is executed during operation,

Program code stored in the memory which executes the method in accordance with one of the previous method claims during operation of the X-ray device.

### BRIEF DESCRIPTION OF THE DRAWINGS

The invention is explained below in greater detail with the aid of the figures, with only the features necessary for understanding the invention being shown. The following reference characters are used: A: Anode; D: Detector; E₁, E₂: Imaging plane; F₁, F₂: Spring focus; O: Object; S_{1,1}-S_{1,3}: First ray bundle; S_{2,1}-S_{2,3}: Second ray bundle; TO₁-TO₃: Subobject.

The individual figures show:

FIG. 1 a schematic diagram of an X-ray of an object under examination and creation of pixel images each with different positions of a spring focus and

FIG. 2 a schematic diagram of an X-ray of an object under examination and transmission of corresponding image pixels relating to an imaging plane in the object under examination.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

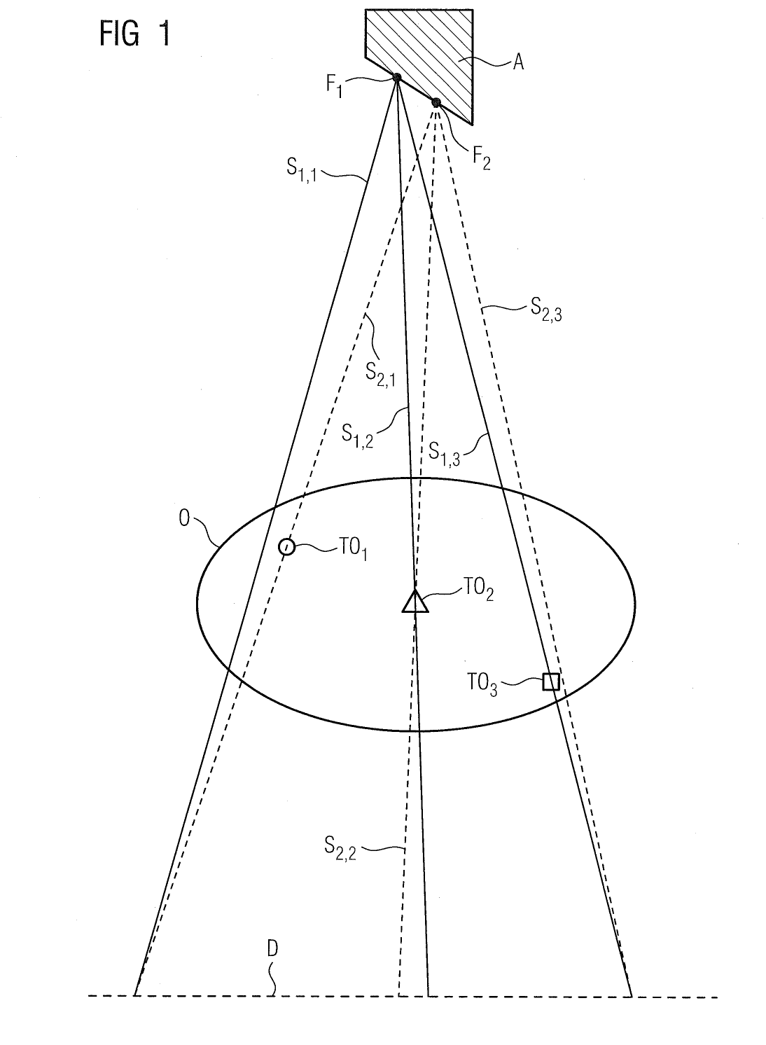

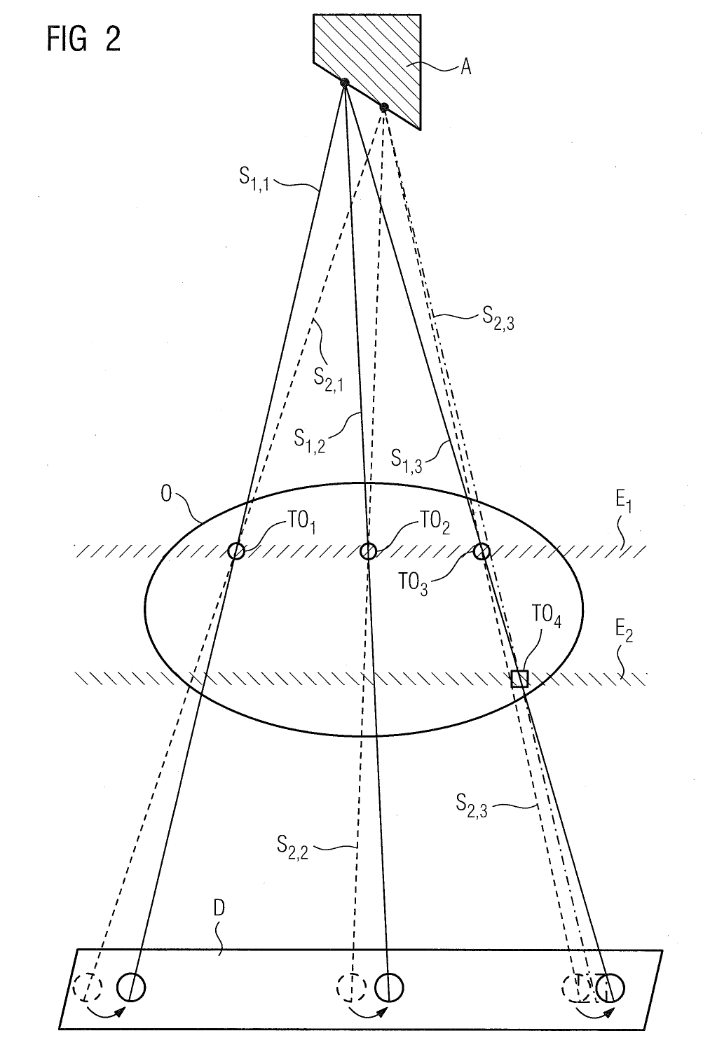

The basic principle of the inventive method will be illustrated in FIGS. 1 and 2. FIG. 1 is a schematic diagram of an X-ray device for recording projectional X-ray images with an anode A, on which are a first spring focus F₁ with an outgoing ray bundle with the rays S_{1,1} to S_{1,3} shown representatively. The ray bundle penetrates an object O with subobjects TO₁ to TO₃ distributed therein, which are arranged at different distances from the focus. The ray bundle thereafter strikes a detector D lying behind the object O, on which the X-rayed object is displayed as a result of its absorption characteristics.

In addition to the first focus F₁ a second focus F₂ is also provided which can be operated alternately with the first focus, so that through the second ray bundle with the representatively shown rays S_{2,1} to S_{2,3} an image of the object O can likewise be recorded with the detector D. It should be noted in this case that, because of the spatially-offset focus positions, the same points in object O—here represented by the three subobjects TO₁ to TO₃—are imaged on the detector by the two ray bundles at different points in each case.

However the option also exists, through simple geometrical consideration for each volume point in the object, of determining from the two ray bundles the ray pair which maps this one point on the detector. Thus for each point in the object a transmission function can be specified which in each case combines the pixels on the detector which pass through specific voxels in the object in each case. This transmission function is however directly dependent on the distance of the respectively observed point in the object. Thus such an operation creates on the two pixel images obtained with the different X-ray bundles a new projection image which in each case provides a “sharp” image of the subobjects at a specific distance. As a rule one will thus select a predetermined plane—which does not have to run absolutely parallel to the detector plane—for which the transmission function will be computed. As a result a number of projection images can be computed from the two pixel images recorded with different ray bundles, which in each case in different planes—if necessary also curved planes—show a sharp image of the subobjects lying in said planes.

FIG. 2 is shown to illustrate how a first ray bundle with three outgoing rays S_{1,1} to S_{1,3} from the first focus intersects three subobjects TO₁ to TO₃ in an imaging plane E₁ and maps them on the detector D. At the same time the dashed line rays S_{2,1} to S_{2,3} show the position of these subobjects TO₁ to TO₃ on the detector D through the second ray bundle with the rays S_{2,1} to S_{2,3}. An embodiment of the inventive determination and application of the transmission function enable an overlay of the two pixel images each recorded with different focus positions to undergo interpolation or weighted interpolation to form a common projection image.

If the subobject TO₃ which lies in another imaging plane E₂ is considered, it is recognized that although a beam S_{1,3} also runs through this subobject, the transmission function which transfers the second image of the subobject TO₃ through the beam S_{2,3} optimally to the first image and thereby shows it sharp produces an incorrect transformation so that this object will be reproduced unsharp.

According to the basic knowledge described above the inventors propose, in their general description, at least one embodiment of a method for creating an X-ray projection image of a three-dimensional object under examination and displaying the projection image, whereby pixel images are recorded from two different perspectives and a projection image is created by overlaying the two pixel images, in that pixel-by-pixel the perspective-related offset of the image pixels to be imaged is taken into account in relation to an imaging surface in the object under examination.

In concrete terms, at least one embodiment of this method can carry out the following method steps:

First radioscopy of the object under examination by X-rays from a first focus position of a first focus and determination of a first pixel image in an image plane, whereby each pixel reflects the attenuation of an X-ray between first focus and pixel through the object under examination,

Second radioscopy of the unchanged object under examination by X-rays from a second focus position of a second focus and determination of a second pixel image in the image plane, whereby each pixel reflects the attenuation of an X-ray between second focus and pixel through the object under examination,

Creation of at least one projection image from the two pixel images by:

Defining an imaging surface which is arranged between pixel image and the focus position and has a rastering with a plurality of raster points,

Determining a transmission function which in each case overlays the image pixels of the first and second pixel image to form a new projection image, the imaging rays of which pass through the same raster point in the imaging surface,

Overlaying the two pixel images with the transmission function to form a new projection image and

Outputting the projection image as an X-ray image of the object under examination with an output device, for example a screen or printer.

In order to achieve as photo-realistic an impression of the projection image as possible, i.e. an impression that the image had been recorded with an optical lens, it is advantageous for an imaging plane to be used as the imaging surface, especially when the imaging plane is aligned in parallel to the pixel images. It is pointed out that there can also be reasons for placing the image plane intentionally at an angle in the examined object, for example in order to show specific subobjects at different depths together in sharp focus.

It is further proposed that a settings controller be used in order to manually move the position of the imaging plane at least in the local area of the object under examination therewith and to view the projection images computed for this purpose as a function of the position of the imaging plane. The corresponding projection images can in this case be computed in real time in each case or made available from a previously computed archive.

Basically the spatial distribution of the pixels in the pixel images used is not of importance, however computations are made easier if the pixel images used for creation of a projection image are identical as regards their positioning of the image pixels.

In at least one embodiment, the inventors further propose, to create the two pixel images, that an X-ray tube with spring focus with at least two focus positions spaced apart from one another be used or alternately that a CT system be used, whereby the projectional pixel images are recorded at two focus positions spaced apart from one another.

The inventors further propose a specific embodiment of the inventive method such that:

A number of projection images are created for a number of spaced-apart imaging planes,

In each projection image unsharp image information is filtered out so that only the sharp image information belonging to the respective imaging plane remains behind, and

From the filtered projection images arranged in accordance with their imaging plane, a 3D arrangement of sectional images and/or a 3D presentation is created.

In addition to the inventive method, an X-ray device for creating an X-ray projection image of a three-dimensional object under examination and displaying the projection image is proposed, comprising:

At least one X-ray tube for radioscopy of the object under examination by X-rays from at least two focus positions and creation of at least two pixel images,

A programmable control and processing unit with a memory for storing program code which is executed during operation,

Program code stored in the memory which executes the method in accordance with one of the previous method claims during operation of the X-ray device.

This X-ray device of at least one embodiment can also have an image output device and a settings controller with which the position of the imaging plane can be moved at least in the local area of the object under examination and the projection images computed for this purpose can be shown with the image output device as a function of the position of the imaging plane.

It goes without saying that the features of the invention specified here are able to be used not only in the respectively specified combination but also in other combinations or on their own, without departing from the framework of the invention.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for creating a projection image of a three-dimensional object under examination, comprising:
recording pixel images from two different perspectives; and
creating the projection image by overlaying the pixel images, a perspective-related offset of the imaging pixels being taken into account, pixel-by-pixel, in relation to an imaging surface in the three-dimensional object under examination.

2. The method as claimed in claim 1, wherein the recording of pixel images includes:
performing a first radioscopy of the object under examination by X-rays from a first focus position of a first focus and determining a first pixel image of the pixel images in an image plane, whereby each respective pixel of the first pixel image reflects an attenuation of an X-ray between the first focus and the respective pixel through the object under examination, and
performing a second radioscopy of the unchanged object under examination by X-rays from a second focus position of a second focus and determining a second pixel image of the pixel images in the image plane, whereby each respective pixel of the second pixel image reflects an attenuation of an X-ray between the second focus and the respective pixel through the object under examination, and
wherein the creating of the projection image from the two pixel images includes:
defining an imaging surface, arranged between the two pixel images and the two focus positions and includes a rastering with a plurality of raster points,
determining a transmission function, which in each case overlays the image pixels of the first and second pixel image to form a new projection image, the imaging rays of which pass through the same raster point in the imaging surface,
overlaying the two pixel images with the transmission function to form a new projection image, and
outputting the projection image as an X-ray image of the object under examination with an output device.

3. The method as claimed in claim 1, wherein an imaging plane is used as the imaging surface.

4. The method as claimed in claim 1, wherein the imaging plane is aligned in parallel to the pixel images.

5. The method as claimed in claim 1, wherein the positioning of the image pixels of the pixel images is identical.

6. The method as claimed in claim 2, wherein a settings controller is used, with which the position of the imaging plane is movable at least in the local area of the object under examination and the wherein projection images computed for this are displayable as a function of the position of the imaging plane.

7. The method as claimed in claim 1, wherein, for creating the two pixel images, an X-ray tube with spring focus with at least two focus positions spaced apart from one another is used.

8. The method as claimed in claim 1, wherein, for creating the pixel images, a CT system is used which records projectional pixel images at two focus positions spaced apart from one another.

9. The method as claimed in claim 2, wherein:
a number of projection images are created for a number of spaced-apart imaging planes,
in each projection image, unsharp image information is filtered out so that only the sharp image information belonging to the respective imaging plane remains behind, and
from the filtered projection images, arranged in accordance with their imaging plane, at least one of a 3D arrangement of sectional images and a 3D representation is created.

10. An X-ray device for creating an X-ray projection image of a three-dimensional object under examination, comprising:
at least one X-ray tube for radioscopy of the object under examination by X-rays from at least two focus positions and creation of at least two pixel images;
a programmable control and processing unit including a memory for storing program code which is executed during operation, the program code stored in the memory, when executed during operation of the X-ray device, causing the programmable control and processing unit to
record pixel images from two different perspectives, and
create the X-ray projection image by overlaying the pixel images, a perspective-related offset of the imaging pixels being taken into account, pixel-by-pixel, in relation to an imaging surface in the three-dimensional object under examination.

11. The X-ray device as claimed in claim 10, wherein an image output device and a settings controller are provided with which the position of the imaging plane is movable at least in the local area of the object under examination and the projection images computed for this purpose are displayable with the image output device as a function of the position of the imaging plane.

12. The method as claimed in claim 2, wherein an imaging plane is used as the imaging surface.

13. The method as claimed in claim 2, wherein the imaging plane is aligned in parallel to the pixel images.

14. The method as claimed in claim 2, wherein the positioning of the image pixels of the pixel images is identical.

15. The method as claimed in claim 2, wherein, for creating the two pixel images, an X-ray tube with spring focus with at least two focus positions spaced apart from one another is used.

16. The method as claimed in claim 2, wherein, for creating the pixel images, a CT system is used which records projectional pixel images at two focus positions spaced apart from one another.

17. A tangible computer readable medium including program segments for, when executed on a computer device, causing the computer device to implement the method of claim 1.

18. A tangible computer readable medium including program segments for, when executed on a computer device, causing the computer device to implement the method of claim 2.
